(19) United States
(12) Patent Application Publication
(10) Pub. No.: US 2015/0133369 A1
(43) Pub. Date: May 14, 2015

(54) PHARMACOKINETIC MODULATION WITH ALISPORIVIR

(71) Applicants: Steven Kovacs, Randolph, NJ (US); June Ke, Warren, NJ (US)

(72) Inventors: Steven Kovacs, Randolph, NJ (US); June Ke, Warren, NJ (US)

(21) Appl. No.: 14/398,817

(22) PCT Filed: May 6, 2013
(86) PCT No.: PCT/US2013/039622

§ 371 Date: Nov. 4, 2014

Related U.S. Application Data
(60) Provisional application No. 61/643,545, filed on May 7, 2012.

(51) Int. Cl. A61K 38/12; A61K 45/06

(52) U.S. Cl. CPC A61K 38/12; A61K 45/06
USPC 514/4.3

(57) ABSTRACT

A method of using alisporivir to therapeutically alter the pharmacokinetics of a medication whereby alisporivir has an effect that achieves and preserves similar safety and efficacy of said drugs with lower doses, less frequent administration, or both.

The present disclosure relates to a non-immunosuppressive cyclosporin which binds to cyclophilins, which are cyclophilin inhibitors, in particular to their pharmaceutical use, for example in the treatment of chronic Hepatitis C virus infection, when co-administered with other drugs.

The cyclosporins comprise a class of structurally distinctive, cyclic, poly-N-methylated undecapeptides, commonly possessing pharmacological, in particular immunosuppressive, or anti-inflammatory activity. The first of the cyclosporins to be isolated was the naturally occurring fungal metabolite Ciclosporin or Cyclosporine, also known as cyclosporin A (CsA).

Cyclosporins which bind strongly to cyclophilin but are not immunosuppressive have been identified. PCT/EP 2004/009804, WO 2005/021028, or WO 2006/071619 disclose non-immunosuppressive cyclosporins which bind to cyclophilins have also been found to have an inhibitory effect on Hepatitis C virus (HCV). WO 2006/038088, incorporated herein by reference in its entirety, describes methods and compositions for the use of alisporivir in the treatment of HCV. Alisporivir (DEBO25 or Debio-O25) is a cyclophilin (Cyp) inhibitor.

Anti-HCV activity of alisporivir has been demonstrated in vitro and in vivo. Efficacious treatment of Hepatitis C virus infection can be achieved when using alisporivir, avoiding the side effects of the current standard of care treatment and thus improving patient compliance. Alisporivir has also shown anti-HIV-1 activity and in vitro was approximately 10-fold more potent than CsA. Furthermore, WO2009/042892 describes methods for the use of alisporivir in the treatment of multiple sclerosis; WO2009/098577 describes methods for the use of alisporivir in the treatment of muscular dystrophy;

WO2008/084368 describes methods for the use of alisporivir in the treatment of Ullrich congenital muscular dystrophy.

Patients suffering from disorders frequently also receive more than one therapy. There is a high potential of drug-drug interaction when more than one drug is being administered. Hence in case of drug-drug interactions, contraindication of co-administration, dose adjustment or strict monitoring may be required in patients receiving more than one drug given the risk associated with altered pharmacokinetic and pharmacodynamic profiles resulting in either diminished therapeutic effect, toxicity or both.

There remains a need for methods and pharmaceutical compositions for treating disorders that do not impact treatment with other drug to avoid adverse events in subjects.

We have found out that cyclophilin inhibitors, in particular alisporivir, can be used effectively for treating patients in need of alisporivir therapy whereby the risk associated with altered pharmacokinetic and pharmacodynamic profiles of a medication administered to same patient is prevented.

### SUMMARY OF THE DISCLOSURE

The present invention generally relates to methods of using alisporivir to therapeutically alter the pharmacokinetics of a medication whereby alisporivir has an effect that achieves and preserves similar safety and efficacy of said drugs and/or alisporivir with lower doses, less frequent administration, or both.

In some aspects, the invention provides methods of treating patients in need of alisporivir therapy whereby the risk associated with altered pharmacokinetic and pharmacodynamic profiles of a medication administered to same patient is prevented.

In other aspect, the present invention provides methods of administering alisporivir to a patient in need of alisporivir therapy or alisporivir for use in treating a patient in need of alisporivir therapy comprising administering to the patient a therapeutically effective amount of alisporivir and coadministering a medication whereby an adverse drug interaction is prevented.

### DETAILED DESCRIPTION OF THE DISCLOSURE

Cytochrome P450 3A4 (CYP3A4) is the most abundant monooxygenase expressed in the liver and is responsible for the oxidative biostransformation of more drugs than any other monooxygenase. It is able to be induced/increased activity and inhibited/decreased activity by a drug or constituents of certain herbal medicines, such as St John's Wort. The effects in both cases have been established as the cause of several, clinically important drug-drug interactions. CYP3A4 is also present in other organs and tissues of a body where it may play a role in metabolism.

P-glycoprotein (P-gp), also known as MDR1 and encoded by the gene ABCB1, is a member of the ATP-binding cassette (ABC) transporter superfamily of transport proteins. Its natural function is to mediate the efflux of compounds from cells. It is able to be inhibited, an effect that has been established as the cause of several clinically important drug-drug interactions.

The organic anion transporting polypeptides (OATP) 1B1 and 1B3, encoded by the genes SLCO1B1 and SLCO1B3, respectively, are members of the solute carrier (SLC) superfamily of transport proteins. The natural function of OATP1B1 and OATP1B3 is to mediate the uptake of both endogenous and exogenous (e.g., xenobiotics) compounds into cells. It is able to be inhibited, an effect that has been established as the cause of several clinically important drug-drug interactions.

Multidrug resistance protein 2 (MRP2), also known as cMOAT and encoded by the gene ABCC2, is a member of the ATP-binding cassette (ABC) transporter superfamily of transport proteins. Its natural function is to mediate the efflux of compounds from cells. It is known to be genetically polymorphic and is able to be inhibited, the latter which has been established as the cause of several clinically important drug-drug interactions.

Xenobiotic substrates of CYP3A4 include macrolide antibiotics (e.g., erythromycin, clarithromycin, telithromycin), benzodiazepines (e.g., alprazolam, diazepam, midazolam, triazolam), cyclosporin A, tacrolimus, HIV antivirals (e.g., indinavir, nelfinavir, ritonavir, saquinavir), calcium channel blockers (e.g., amlodipine, nifedipine, felodipine, diltiazem, verapamil, nitrendipine, nisoldipine), HMG CoA reductase inhibitors (e.g, atorvastatin, cerivastatin, lovastatin, simvastatin), buspirone, dapsone, dexamethasone, haloperidol, ondansetron, propranolol, sildenafil, trazodone, and zolpidem.

Xenobiotic substrates of P-gp include digoxin, fexofenadine, loperamide, paclitaxel, doxorubicin, vinblastine, and irinotecan.

Xenobiotic substrates of OATP1B1 and/or OATP1B3 include HMG CoA reductase inhibitors (e.g., atorvastatin, colloquially “statins”), valsartan, telmisartan, olmesartan, repaglinide, digoxin, and fexofenadine.

Xenobiotic substrates of MRP2 include valsartan, olmesartan, methotrexate, etoposide, mitoxantrone, and glutathione and glucuronide conjugates.

Xenobiotic substrates of BSEP include taurocholic acid and pravastatin.

Therapeutic alteration of one drug's pharmacokinetics and general in vivo disposition by alisporivir, which is mediated by inhibition of CYP3A4, P-gp, OATP1B1, OATP1B3, MRP2, BSEP, and/or NTCP by alisporivir, results in increased blood or plasma concentrations and/or decreased clearance of said drug such that lower doses, less frequent administration, or both can achieve and preserve the safety and efficacy of said drug.

CYP3A4 inhibitors and inducers, sensitive CYP3A4 substrates, CYP3A4 substrates with narrow therapeutic ranges/windows, and drugs that are substrates for both CYP3A4 and uptake and efflux transporters such as OATP1B1, OATP1B3, MRP2, BSEP, NTCP, and P-gp, are contraindicated for use with alisporivir. Specific examples of contraindicated drugs include HMG-CoA reductase inhibitors and PDE5 inhibitors.

As used herein, a strong inhibitor for a specific CYP3A4 is defined as an inhibitor that increases the AUC (area under the curve) of a substrate for that CYP3A4 by equal or more than about 5-fold.

As used herein, a moderate inhibitor for a specific CYP3A4 is defined as an inhibitor that increases the AUC of a sensitive substrate for that CYP3A4 by less than about 5-fold but equal to or more than about 2-fold.

As used herein, a weak inhibitor for a specific CYP3A4 is defined as an inhibitor that increases the AUC of a sensitive substrate for that CYP3A4 by less than about 2-fold.

As used herein, “microgram/kilogram” means microgram drug per kilogram body weight of the mammal—including man—to be treated.

As used herein, the term “treatment” or “treat” refer to both prophylactic or preventative treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

By “therapeutic regimen” is meant the pattern of treatment of an illness, e.g., the pattern of dosing used during HCV therapey. A therapeutic regimen may include an induction regimen and a maintenance regimen.

The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both.

The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “about”, unless the context dictates otherwise, is used to mean a range of +or −10%.

As used herein “up to 12, 24, 48 or 72 weeks” refers to the treatment duration and is intended to mean for about 12 weeks, about 24 weeks, about 48 weeks, or about 72 weeks, respectively. It will be understood that therapy need not end at exactly the 12, 24, 48 or 72 week time period. For example, therapy may end a day or a few days before the 24 week period, and still be an equivalent within the scope and spirit of the current disclosure.

As used herein “twice per day” or BID means twice in any period of about 24 hour period; “once per day” or QD means once in any period of about 24 hour period; “once per week” is used to mean once in any period of about seven days.

As used herein pharmacokinetics include the time course of in vivo exposure to a drug and any parameter that may describe the same (e.g., half-life, time to peak plasma concentration, peak plasma concentration, AUC, clearance, volume of distribution, etc.)

As used herein AUCinf refers to Area Under the Curve infinity in [hr ng/mL] indicating the integrated quantity of analyte or drug (the serum concentration curve) after dosing.

As used herein AUClast refers to Area Under the Curve last sample in [hr ng/mL] where activity could be detected.

As used herein Cmax refers to the maximum concentration of the analyte or drug in [ng/mL] achieved after dosing.

As used herein a pharmacokinetic improving or blood plasma level increasing effective amount of alisporivir

A method of coadministration of alisporivir with one or more drugs that have its pharmacokinetics and general in vivo disposition affected by at least one of CYP3A4, P-gp, OATP1B1, OATP1B3, MRP2, BSEP, and NTCP wherein alisporivir has an effect that achieves and preserves similar safety and efficacy of said drugs with lower doses, less frequent administration, or both.

A method for improving the pharmacokinetics or increasing blood plasma levels of a drug which is metabolized by cytochrome P450 monooxygenase, comprising administering to a patient treated with said drug, a pharmacokinetic improving or blood plasma level increasing effective amount of alisporivir. Alisporivir may be administered prior to, and/or substantially contemporaneously with, a drug wherein efficacy of said drug is compromised due to degradation by cytochrome P450 monooxygenase.

Yet another embodiment of the invention is a method for improving the pharmacokinetics or increasing blood plasma levels of a drug, comprising administering to a patient treated with said drug, a pharmacokinetic improving or blood plasma level increasing effective amount of alisporivir wherein the drug is a P-glycoprotein and/or cytochrome P450 monooxygenase 3A4 substrate.

A method for improving the pharmacokinetics or increasing blood plasma levels of a drug, comprising co-administering to a patient being treated with said drug, a pharmacokinetic improving or blood plasma level increasing effective amount of alisporivir wherein the drug is a P-glycoprotein and cytochrome P450 monooxygenase 3A4 substrate.

Method of co-administration of alisporivir with one or more compounds which is metabolized by cytochrome P450 monooxygenase 3A4.

Method of co-administration of alisporivir with one or more compounds which is metabolized by cytochrome P450 monooxygenase 3A4 comprising orally administering alisporivir and wherein a dose reduction or a clinical monitoring of a patient to whom the compound is concomitant administered or a contraindication for co-administration for the compound should be considered.

Method of co-administration of alisporivir with one or more compounds which is/are a substrate of at least one of the following P-glycoprotein, OATP1B1, MRP2, BSEP or NTCP, comprising orally administering alisporivir and wherein a dose reduction or a clinical monitoring of a patient to whom the compound is concomitant administered or a contraindication for co-administration for the compound should be considered.

Method of co-administration of alisporivir with valsartan comprising orally administering alisporivir and wherein dose reduction for valsartan should be considered.

Method of co-administration of alisporivir with methadone comprising orally administering alisporivir and wherein dose reduction for methadone should be considered.

Method of administering alisporivir to a patient in need of alisporivir therapy or alisporivir for use in treating a patient in need of alisporivir therapy comprising administering to the patient a therapeutically effective amount of alisporivir and co-administering a medication whereby an adverse drug interaction is prevented.

Methods of treating patients in need of alisporivir therapy whereby the risk associated with altered pharmacokinetic and pharmacodynamic profiles of a medication administered to same patient is prevented.

In another embodiment, alisporivir may be administered with additional agents of the standard of care for treatment of chronic Hepatitis C that promote the antiviral efficacy of the therapy treatment. Additional agents that promote the antiviral efficacy of the therapy treatment including direct acting antiviral agents such as polymerase inhibitors, protease inhibitors, substrate-based protease inhibitors of HCV NS3-4A serine protease, non-substrate-based NS3 protease inhibitors; phenanthrenequinones, thiazolidines and benzanilides, nucleosides analogs, antisense molecules directed against HCV genome or any cellular component that is required for viral replication, vaccine or antibody-based approaches to HCV treatment.

Direct acting antiviral agents, is used herein to mean agents that interfere with specific steps in the hepatitis C virus (HCV) replication cycle. Such agents may be, e.g., ribavirin derivatives, protease inhibitors, and polymerase inhibitors (e.g., nucleoside and non-nucleoside inhibitors). Exemplary direct acting antiviral agents include: boceprevir, telaprevir, ABT-072, ABT-450, ABT-333 by Abbott, ACH1625 by Achillion, ANA598 by Anadys Pharmaceuticals, AZD-7295 by AstraZeneca, BI201335, BI207127 by Boehringer Ingelheim Pharma, BMS650032, BMS790052, BMS791325, BMS824383 by Bristol Myers Squibb, Clemizole by Eiger BioPharmacetucials, Filibuvir by Pfizer, GS9190 (Tegobuvir), GS9256 by Gilead, IDX375 by Idenix, INX-189 by Inhibitex, PSI-7851, PSI-938 by Pharmasset, PSI-7977, RG7128 by Pharmasset/Genethec, PPI-461 by Presidio RG7227 (Danoprevir) by InterMune/Genentech, SCH900518 (Narlaprevir), Vaniprevir by Merck, TMC435 by Medivir/Tibotec, VX-222, VX-759, VX-500, VX-916 by Vertex.

In another embodiment the present invention further describes methods of treating patients in need of alisporivir therapy whereby the risk associated with altered pharmacokinetic and pharmacodynamic profiles of a medication administered to same patient is prevented, wherein the medication administered to same patient is one or more direct acting antiviral selected from boceprevir, telaprevir, ACH1625, ACH2684, INX-189, INX-184, Danoprevir, Daclastavir (also known as BMS-790052), ABT-450.

In the above embodiments and throughout this specification, the standard of care treatment is a treatment that is used to treat chronic Hepatitis C infections. The current standard of care treatment for all genotypes includes administration of interferon, in particular pegylated interferon in combination with ribavirin. Specifically for genotype 1 HCV, the standard of care includes a third drug in addition to pegylated interferon and ribavirin, which is currently an NS3/4A protease inhibitor, currently either telaprevir or boceprevir.

In one embodiment, the present invention further provides alisporivir for use in combination with standard of care in treatment of a Hepatitis C virus infected patient, the alisporivir to be administered in an amount of about 200 to about 600 mg (e.g., about 200 mg, about 350 mg, about 400 mg, about 450 mg, about 500 mg, about 550 mg, about 600 mg, about 650 mg) once or twice per day.

In still other aspects, the present invention provides a package comprising instructions to administer alisporivir according to any method described herein.

In treatment described above effective dosages of the standard of care agents are administered in compositions, i.e. they may be administered together (i.e., simultaneously), but may also be administered separately or sequentially. In general, combination therapy is typically administered together, the rationale being that such simultaneous administration induces multiple simultaneous stresses on the virus. The specific dosages given will depend on absorption, inactivation and excretion rate of the drugs as well as other factors. It is to be noted that dosage values will also vary with the severity of the condition to be alleviated.

The terms “co-administration” or “combined administration” or “administered in combination with” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present invention. The administration of a pharmaceutical combination of the invention results in a beneficial effect, e.g. a synergistic or additive therapeutic effect, compared to a monotherapy applying only one of its pharmaceutically active ingredients or as compared to the current standard of care therapy. The treatment used in the methods described herein may be administered by any conventional route. One or more components may be administered parentally, e.g., in the form of injectable solutions or suspensions, or in the form of injectable deposit formulations. Preferably, alisporivir will be administered orally in the form of solutions or suspensions, tablets or capsules. Pharmaceutical compositions for oral administration comprising alisporivir typically further comprise one or more pharmaceutically acceptable carrier substances. Typically, these compositions are concentrated and need to be combined with an appropriate diluent, e.g., water, prior to administration. Pharmaceutical compositions for parenteral administration typically also include one or more excipients. Optional excipients include an isotonic agent, a buffer or other pH-controlling agent, and a preservative. These excipients may be added for maintenance of the composition and for the attainment of preferred ranges of pH (about 6.5-7.5) and osmolarity (about 300 mosm/L).

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer stem cell. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration.

The efficacy of a therapy regimen may be monitored using standard protocols.

In case of HCV therapy, treatment may be followed by determinations of HCV in serum and measurement of serum ALT levels. For example, the patients may be assessed for the presence of HCV RNA in their plasma. HCV RNA (IU/mL) can be measured at regular intervals during the treatment, e.g., at Day 1 (pre-dose and 4, 8, and 12 hours post-dose) and pre-dose at Day 2, Day 3, Day 8, Day 15, Day 29, and at Week 12, Week 24, Week 36, Week 48, Week 72 (when applicable), and at follow up. In addition, the HCV strains in the patient can be sequenced and assessed for identification of mutations selecting for resistance. The endpoint of treatment is a virological response, i.e., the absence of HCV at the end of a treatment course, several months after initiation of treatment, or several months after completion of treatment. HCV in serum may be measured at the RNA level by methods such as quantitative RT-PCR or northern blots or at the protein level by enzyme immunoassay or enhanced chemiluminescence immunoassay of viral proteins. The endpoint may also include a determination of a serum ALT level in the normal range.

HCV RNA levels can be measured using commercially available methods. Individual methods may have their own unique LOD and LOQ. As used herein, LOD means limit of detection and LOQ means limit of quantification of HCV RNA levels for the method. For example, when using the COBAS® TaqMan® HCV Test, v2.0 (Roche Diagnostics) for assessment of HCV RNA levels, LOQ of 25 IU/ml (1.398 log10) and LOD of 10 IU/ml (1 log10) have been reported.

The following Examples illustrate the invention described hereinbefore.

### EXAMPLES

### 1. Interaction with CYP3A4 and Cellular Uptake and Efflux Transporters

The results of the in vitro studies establish alisporivir as a CYP3A4 substrate and time-dependent inhibitor of CYP3A4. As a substrate of CYP3A4, alisporivir may be affected by potent induction or inhibition of CYP3A4 by other drugs and food, such as for example grapefruit juice. Alisporivir is also an inhibitor of P-gp, OATP1B1, OATP1B3, MRP2, BSEP, and NTCP in vitro. Such inhibitory effects have the potential to cause clinically significant interactions with other drugs in vivo. Alisporivir has no inducing effects on CYP isozymes in vitro and does not inhibit UGT1A1 or UGT2B7.

### 2. Effects of Alisporivir on Other Drugs

Open label, single dose, randomized, crossover clinical studies have been performed with alisporivir and various other drugs.

Buspirone is an antianxiety agent with an unknown mechanism of action. It exhibits high affinity for serotonin (5-HT(1A)) receptors, moderate affinity for brain D(2)-dopamine receptors and no significant affinity for benzodiazepine receptors. It has no effect on GABA binding. Additional details can be found in the prescribing information for buspirone.

Buspirone 5 mg is the lowest available formulation strength and is one-third of the recommended starting dose (15 mg) and therefore allows the largest margin of safety for possible increased exposure caused by alisporivir-mediated inhibition of CYP3A4. The highest recommended clinical dose of buspirone is 60 mg usually divided into two to three doses. Buspirone bioavailability is increased with food and in order to minimize any risks, buspirone will be administered on an empty stomach (˜1 hour before breakfast).

Digoxin is a cardiac glycoside that inhibits sodium-potassium ATPase, which increases intracellular sodium concentration leading to increased intracellular calcium concentration.

Autonomic effects of this include vagomimetic action and baroreceptor sensitization which lead to positive inotropic action, reduced sympathetic response and decreased reninangiotensin system output (neurohormonal deactivation). Additional details can be found in the prescribing information for digoxin.

Digoxin 0.25 mg is generally initiated in patients under the age of 70 with good renal function. When digoxin is taken with food high in bran fiber, the amount absorbed from an oral dose may be reduced. In order to get less variable exposure, digoxin will be administered on an empty stomach (˜1 hour before breakfast) for consistency in each part.

Valsartan is a nonpeptide angiotensin II antagonist that selectively blocks the binding of angiotensin II to the AT 1 receptors in tissues such as vascular smooth muscle and the adrenal gland. In the renin-angiotensin system, angiotensin I is converted by angiotensin-converting enzyme (ACE) to form angiotensin II. Angiotensin II stimulates the adrenal cortex to synthesize and secrete aldosterone, which decreases the excretion of sodium and increases the excretion of potassium. Angiotensin II also acts as a vasoconstrictor in vascular smooth muscle. Valsartan, by blocking the binding of angiotensin II to the AT 1 receptors, promotes vasodilation and decreases the effects of aldosterone. The negative feedback regulation of angiotensin II on renin secretion also is inhibited, resulting in a rise in plasma renin concentrations and a consequent rise in angiotensin II plasma concentrations; however, these effects do not counteract the blood pressure-lowering effect that occurs.

Valsartan 40 mg is the lowest recommended daily or individual dose for initiating therapy for post-myocardial infarction care (20 mg BID) and heart failure (40 mg BID), respectively.

The dose of 40 mg is also half of the recommended starting dose for the treatment of hypertension (80 mg/day). The highest recommended dose for clinical use is 320 mg/day, which has previously been shown to have no blood pressure-lowering effect in normotensive subjects. Thus, a single dose of 40 mg provides an appropriate safety margin should exposure be increased by alisporivir. Valsartan bioavailability is decreased with food and therefore, valsartan will be administered on an empty stomach (˜1 hour before breakfast).

Drug concentrations in plasma and serum was determined using a validated LC-MS/MS method.

The ratio of buspirone/digoxin/valsartan AUClast, AUCinf and Cmax ratio without and with co-administration of alisporivir were analyzed by a linear mixed effect model. Separate analyses were fitted for each drug and PK parameter.

There were no serious adverse events or discontinuations due to adverse events.

Alisporivir is confirmed to have independent and clinically important inhibitory effects on CYP3A4, P-gp (ABCB1), MRP2 (ABCC2), and OATP1B1 (SLCO1B1) in vivo.

Buspirone (A Sensitive CYP3A4 Substrate)

Alisporivir 600 mg BID for 7 days caused an approximate 6.6-fold increase in the AUC and 4.5-fold increase in the Cmax for buspirone following a single oral 5 mg dose of buspirone. The AUC for the metabolite of buspirone, 1-PP, increased by approximately 12-15%.

Digoxin (A P-gp Substrate)

Alisporivir 600 mg BID for 7 days caused an approximate 2.6-fold increase in the AUC and an approximate 1.8-fold increase in Cmax for digoxin following a single oral 0.25 mg dose of digoxin. Alisporivir did not affect the renal clearance of digoxin

Valsartan (a CYP2C9, OATP1B1, OATP1B3, and MRP2 Substrate)

Alisporivir 600 mg BID for 7 days caused an approximate 6-fold increase in the AUC for valsartan and an approximate 4-fold increase in the AUC for 4-hydroxyvalsartan following a single oral 40 mg dose of valsartan. The Cmax of valsartan and 4-hydroxyvalsartan increased approximately 2.3- and 2-fold, respectively.

Atorvastatin (A CYP3A4 and OATP1B1 Substrate)

Alisporivir 600 mg BID for 10 days caused an approximate 21- to 23-fold increase in exposure (AUC and Cmax) to atorvastatin and p-OH-atorvastatin following a single 40 mg oral dose of atorvastatin. The Cmax and AUC for o-OH-atorvastatin (another hydroxylated metabolite) increased approximately 7- and 9-fold, respectively. The T1/2 of atorvastatin and its metabolites were similar when atorvastatin was administered alone or with alisporivir.

Azithromycin (A CYP3A4 Substrate)

A single 1200 mg oral dose of alisporivir caused an approximate 32-39% increase in the AUC of azithromycin following a single oral 1000 mg dose of azithromycin.

Telaprevir (A CYP3A4 and P-gp Substrate)

In healthy human subjects, alisporivir 600 mg BID for 7 day and 600 mg QD for one additional day caused an approximate 3.2- to 3.6-fold increase in the AUC and an approximate 2.4-fold increase in the Cmax of telaprevir following a single 375 mg oral dose of telaprevir. The T1/2 of telaprevir was longer when telaprevir was administered with alisporivir.

In healthy human subjects, telaprevir 750 mg TID for 10 days caused an approximate 7.5- to 7.8-fold increase in the AUC and 3.6-fold increase in the Cmax of alisporivir following a single 600 mg oral dose of alisporivir. The T1/2 of alisporivir was shorter when alisporivir was administered with telaprevir.

Boceprevir (A CYP3A4 and P-gp Substrate)

In healthy human subjects, alisporivir 600 mg BID for 7 days and 600 mg QD for one additional day caused an approximate 43 to 54% increase in the AUC and an approximate 47% increase in the Cmax of boceprevir following a single 400 mg oral dose of boceprevir. The T1/2 of boceprevir was similar when boceprevir was administered with alisporivir.

In healthy human subjects, boceprevir 800 mg TID for 4 days caused an approximate 3.3- to 3.4-fold increase in the AUC and 2-fold increase in the Cmax of alisporivir following a single 600 mg oral dose of alisporivir. The T1/2 of alisporivir was shorter when alisporivir was administered with boceprevir.

Fexofenadine (P-gp Substrate, May be a Substrate of OAT1B1 and BESP)

In healthy human subjects, alisporivir 600 mg BID for 7 day and 600 mg QD for two additional days caused an approximate 8.8- to 9.4-fold increase in the AUC and an approximate 6.6-fold increase in the Cmax of fexofenadine following a single 60 mg oral dose of fexofenadine. The T1/2 of fexofenadine was similar when fexofenadine was administered with alisporivir.

Escitalopram (A Substrate for CYP2C19 (37%), CYP3A4 (35%), and CYP2D6 (28%)

In healthy human subjects, alisporivir 600 mg BID for 7 day and 600 mg QD for three additional days caused an approximate 1.3-fold increase in the AUC and an approximate 0.83-fold change (17% decrease) in the Cmax of escitalopram following a single 10 mg oral dose of escitalopram. The T1/2 of escitalopram was similar when escitalopram was administered with alisporivir.

Fluvastatin (A Substrate CYP2C9, OATP1B1/1B3 and BSEP)

In healthy human subjects, alisporivir 600 mg BID for 7 day and 600 mg QD for one additional day caused an approximate 2.5- to 2.6-fold increase in the AUC and an approximate 2.1-fold increase in the Cmax of fluvastatin following a single 20 mg oral dose of fluvastatin. The T1/2 of fluvastatin was longer when fluvastatin was administered with alisporivir.

### 3. Effect of Alisporivir on Circulating Bilirubin Concentrations in Healthy Subjects

A transient hyperbilirubinemia associated with alisporivir has been observed in healthy subjects and chronic Hepatitis C patients. Reversible drug-related hyperbilirubinemia is known to occur for several drugs by inhibition of bilirubin transport and/or conjugation. In vitro, alisporivir inhibits the uptake transporters OATP1B1 and OATP1B3 (IC50 0.7 μM) and the efflux transporter MRP2 (Ki 5.9 μM) but does not inhibit the conjugating enzyme UGT1A1, all involved in the normal handling of bilirubin. Since ribavirin-associated hemolysis contributes to bilirubin changes that are evident in chronic Hepatitis C patients, the time course of changes in bilirubin associated with the highest intended therapeutic exposure to alisporivir was investigated in healthy subjects to characterize the specific effect of ALV. The effect of Alisporivir on the OATP1B1, 1B3, and MRP2 substrate valsartan was also investigated.

Alisporivir 600 mg BID was administered for 7 days followed by 600 mg QD for 1, 2, or 3 days to three groups of 16 healthy subjects (48 total) in 3 different drug-drug interaction studies. One group received 40 mg valsartan before and after alisporivir. Subjects known or suspected to have any inherited bilirubin disorder were excluded. Laboratory tests performed at baseline, on days 4, 7, 9, 10 /11, and end-of-study (14±1 days after the last dose).

All subjects completed the study and tolerated alisporivir well. Mean change from baseline increases of 9.9-11.3 μM (0.6-0.7 mg/dL; median 2.3-fold) in total bilirubin occurred between days 7 and 9 without coincident changes in other LFTs. Maximum total bilirubin concentration was 46 μM (2.7 mg/dL), observed on Day 9 in a single subject whose baseline was 15 μM (0.9 mg/dL). All bilirubin increases were asymptomatic and reversible. Total bilirubin exceeded the ULN in 4 subjects (8.3%) and an increase in unconjugated (indirect) bilirubin predominated.

Valsartan exposure increased ˜6-fold.

This showed that alisporivir inhibits OATP1B1, OATP1B3, and MRP2 in vivo causing increases in total bilirubin and valsartan.

1. Method of coadministration of alisporivir with one or more drugs that have its pharmacokinetics and general in vivo disposition affected by at least one of CYP3A4, P-gp, OATP1B1, OATP1B3, MRP2, BSEP, and NTCP wherein alisporivir has an effect that achieves and preserves similar safety and efficacy of said drugs and/or alisporivir with lower doses, less frequent administration, or both.

2. The method according to claim 1, wherein said drug is a direct-acting antiviral drug used in the treatment of HCV infection.

3-8. (canceled)

9. Method of co-administration of alisporivir with one or more compounds which is/are a P-glycoprotein, OATP1B1, MRP2, BSEP or NTCP substrate comprising orally administering alisporivir and wherein a dose reduction or a clinical monitoring of a patient to whom the compound is concomitantly administered or a contraindication for co-administration for the compound should otherwise be considered.

10. Method of co-administration of alisporivir according to claim 9 with valsartan comprising orally administering alisporivir and wherein dose reduction for valsartan should be considered.

11. (canceled)

12. Method of administering alisporivir to a patient in need of alisporivir therapy or alisporivir for use in treating a patient in need of alisporivir therapy comprising administering to the patient a therapeutically effective amount of alisporivir and co-administering a medication whereby an adverse drug interaction is prevented.

13. (canceled)
